(19) United States
(12) Patent Application Publication
(10) Pub. No.: US 2007/0112050 A1
(43) Pub. Date: May 17, 2007

(54) HMGCoA reductase inhibitor combinations and uses thereof

(75) Inventors: Paul Ashton, Boston, MA (US); Grazyna Cynkowska, Brookline, MA (US); Tadeusz Cynkowski, Brookline, MA (US); Thomas J. Smith, Pasadena, CA (US)

Correspondence Address: FISH & NEAVE IP GROUP;ROPES & GRAY LLP, ONE INTERNATIONAL PLACE, BOSTON, MA, 02110-2624, US

(73) Assignee: pSivida Inc., Wartertown, MA (US)

(21) Appl. No.: 11/404,042

(22) Filed: Apr. 12, 2006

Related U.S. Application Data
(60) Provisional application No. 60/670,772, filed on Apr. 12, 2005.

(51) Int. Cl. A61K 31/4178; A61K 31/401; A61K 31/366; A61K 31/22

(52) U.S. Cl. 514/381; 514/423; 514/460; 514/548

(57) ABSTRACT

The invention provides a compound comprising a first pharmacological moiety connected to at least a second pharmacological moiety through a physiologically labile linker, or a salt thereof. The invention also provides a method of reducing cardiovascular disease or cardiovascular disease-related conditions in an individual. The method involves administering to an individual with cardiovascular disease an effective amount of a compound, in which the compound has a first pharmacological moiety linked to a second pharmacological moiety, and the compound or either or both of the constituent pharmacological moieties acts to reduce, treat, or prevent cardiovascular disease. The compounds of the invention can be delivered in a drug delivery device.

### CROSS REFERENCE TO RELATED APPLICATIONS

This application claims the benefit of U.S. Provisional Patent Application No. 60/670,772, filed on Apr. 12, 2005, the specification of which is hereby incorporated by reference in its entirety.

### BACKGROUND OF THE INVENTION

Cardiovascular diseases, which include coronary heart disease and stroke, are the leading causes of death in the United States. The major risk factors of cardiovascular diseases are high blood cholesterol, high blood pressure (hypertension), and smoking and dietary factors. Stamler J., Established Major Coronary Risk Factors. In: Coronary Heart Disease Epidemiology: From Aetiology To Public Health, Marmot M & Elliott P, eds., 35-66 (Oxford University Press, New York, 1992). Elevated blood cholesterol is a major risk factor for coronary heart disease, and hypertension is the major risk factor for stroke. Hypertension can also increase the risk of myocardial infarct. Many clinical trials have demonstrated the efficacy of antihypertensive and lipid-lowering drugs for treating cardiovascular diseases. National Institutes of Health. The Sixth Report Of The Joint National Committee On Prevention, Detection, Evaluation, And Treatment Of High Blood Pressure. NIH publication no. 98-4080 (Rockville, Md.: US Department of Health and Human Services, National Institutes of Health, National Heart, Lung, and Blood Institute, November 1997); National Cholesterol Education Program. Second Report Of The Expert Panel On Detection, Evaluation And Treatment Of High Blood Cholesterol In Adults. NIH publication no. 93-3095 (Rockville, Md.: US Department of Health and Human Services, National Institutes of Health, 1993).

However, some important indicators of risk for cardiovascular disease have not improved recently, have leveled off, or are reversing. For example, approximately 70% of persons with hypertension do not have the condition controlled at levels below 140/90 mm Hg, and death rates for stroke have not declined in recent years. National Heart, Lung and Blood Institute. Morbidity & Mortality: 1998 Chartbook On Cardiovascular, Lung, And Blood Diseases. (Rockville, Md.: US Department of Health and Human Services, National Institutes of Health, 1998; Higgins M & Thom T, Int. J. Epidemiol. 1989;18:S58-S66). Heart failure has emerged as a health concern for older adults (CDC, MMWR 47:633-7 (1998)), and adults who survive a myocardial infarction or other hypertension-related diseases remain at increased risk for heart failure.

Medications can only be effective if patients comply with their therapeutic regimen. The problem of patient noncompliance with medication use remains one of the most significant issues facing our health care system. The negative impact of noncompliance on patient outcomes has been documented for patients with hypertension. Morse, G. D. et al., Am. J. Hosp. Pharm. 43:905-909 (1986). Conversely, there is good evidence that patients who are more compliant in taking antihypertensive medications are more likely to achieve blood pressure control. Caro, J. J. & Speckman, J. L., J. Hypertension. 16:S31-S34 (1998).

The availability of several different drug targets for controlling hypertension has offered the potential of multiple-drug regimens. Polypharmacy is difficult to avoid, because using one drug can control blood pressure in only about 50% of patients. However, such multiple-drug regimens reduce patient compliance. Oparil, S. & Calhoun, D. A, American Family Physician, 1007 (Mar. 1, 1998). As a partial solution to this problem, fixed-dose combination therapy is designed to improve patient compliance by decreasing the number of pills that must be taken and reducing the dose-dependent adverse effects of individual components. Sica, D. A., Drugs 48:16-24 (1994). To be combined in a single-dose form, however, U.S. law requires that each component in the combination must contribute to therapeutic effect and that the dosage of each component must be such that the combination is safe and effective in a major proportion of the target population (i.e., patients whose hypertension is not easily controlled with a single drug). 21 C.F.R. § 300.50 (the “fixed combination” policy). See also, 21 C.F.R. § 330.10(a)(4)(iv).

Accordingly, there is a continuing need in the medical arts for pharmaceutical compounds that deliver two or more drugs that are effective for treating cardiovascular disease at a single time in a single dose, to enhance patient compliance, among other advantages.

### SUMMARY OF THE INVENTION

The invention provides a compound comprising a first pharmacological moiety connected to at least a second pharmacological moiety through a physiologically labile linker, or a salt thereof. The first pharmacological moiety is an HMGCoA reductase inhibitor. The second pharmacological moiety is selected from an angiotensin II (AT₁) receptor blocker, a cholesterol absorption blocker, a cholesteryl ester transfer protein inhibitor (or other agent that beneficially affects HDL or LDL levels), or an HMGCoA reductase inhibitor which may be the same or different than the first HMGCoA reductase inhibitor. The two or more pharmacological moieties can be linked either by covalent bonds or by ionic interactions.

The invention also relates to compounds comprising a first pharmacological moiety connected to at least a second pharmacological moiety through a physiologically labile linker, or a salt thereof. The pharmacological moieties are each independently selected from ACE inhibitors, cardioprotective agents, steroids and corticosteroids, sex steroids, apoptosis inhibitors, agents that alter expressions/activity of MMPs, and anti-inflammatory agents. The two or more pharmacological moieties can be linked either by covalent bonds or by ionic interactions.

The invention also provides a method of reducing cardiovascular disease or cardiovascular disease-related conditions in an individual. The method involves administering to an individual with cardiovascular disease an effective amount of a compound, in which the compound has a first pharmacological moiety linked to a second pharmacological moiety, and the compound or either or both of its constituent pharmacological moieties acts to reduce, treat, or prevent cardiovascular disease. The compounds of the invention can be delivered in a drug delivery device.

The use of the compounds of the invention is a convenience for both cardiovascular disease patients and for their physicians. Administration of compounds also encourages improved patient compliance, which improves health.

The use of the compounds of the invention may also be a convenience for the pharmacist because use of the compounds of the invention permits simplified titration processes for drug preparation. Potentially, the cost of prepared compounds can be less than that of preparations of the individual components, after packaging costs are included.

Moreover, the compounds of the invention can reasonably be expected to potentiate the separate cardiovascular effects by additive or synergistic effect. Where such additive or synergistic effects occur, a reduction in adverse events can be achieved through lower dosage requirements of the separate moiety components. In general, an improved overall antihypertensive effect can be achieved where the ratio of the separate moiety components is superior to what is available in the absence of a fixed-dose combination

### BRIEF DESCRIPTION OF THE DRAWINGS

FIG. 1 is a diagram of the renin-angiotensin-aldosterone system.

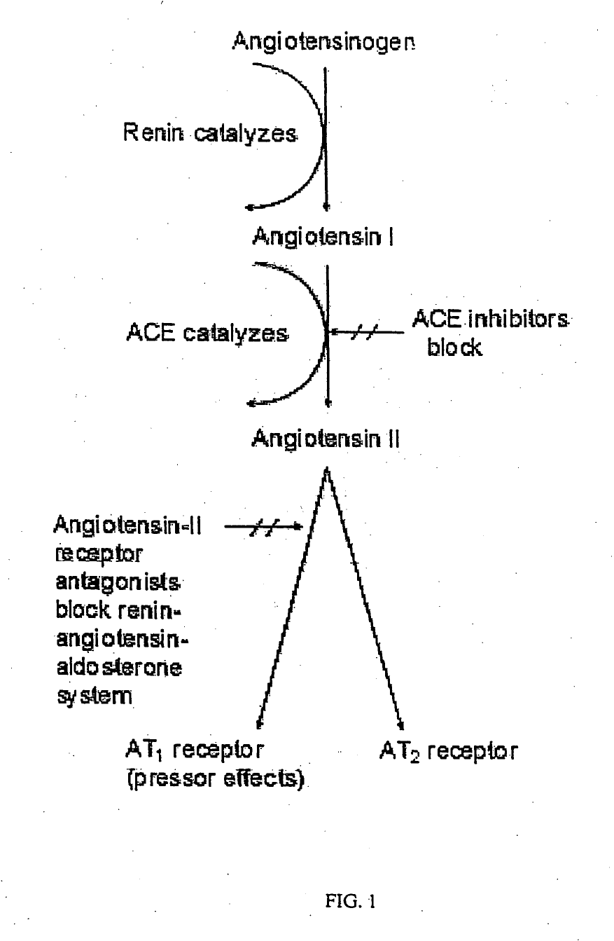

FIG. 2 is a diagram showing the similarity of structure among the HMGCoA reductase inhibitors (from Istvan, E. S. & Deisenhofer, J., Science 292: 1160-64 (2001)). The HMG-moiety is indicated by the dotted box, and the Kₘ value of HMGCoA is indicated. Not shown in this figure are lovastatin (a type I HMGCoA reductase inhibitor) and provastatin (a type II HMGCoA reductase inhibitor).

FIG. 3 is a diagram of a reaction scheme for telmisartan and simvastatin.

FIG. 4 is a diagram of a reaction scheme for telmisartan and lovastatin.

### DETAILED DESCRIPTION OF THE INVENTION

The invention provides a means of improving the pharmacology and delivery properties of pharmacologically active moieties, by conjugating them together to form a new compound. A “pharmacological moiety” is a compound that, when active or when activated, can cause an intended medical effect. Pharmacological moieties typically cause these effects when made to interact with a drug target (generally in the body of the individual to whom the compound is administered, particularly a human or mammal that is a model of a human disease or condition, but possibly also in an animal, such as a bird or mammal, in a veterinary administration of the compound). In this invention, the pharmacological moiety affects hypertension and hypertension-related diseases and conditions in animals, particularly mammals, more particularly, humans. Hypertension-related diseases are known in the medical arts and include damage to the blood vessels of the brain, heart, and kidneys, stroke, cardiac failure, renal failure and an increased risk of myocardial infarct (MI).

A compound of the invention is a composition of at least two pharmacological moieties, either covalently linked to one another by a (usually labile) bond to form a single compound or ionically linked to one another to form a single working composition (see, U.S. Pat. No. 6,051,576, incorporated by reference). The term “codrug” as used herein means a compound, or a prodrug form thereof, comprising a first small molecule residue associated with a second small molecule residue, wherein both residues, in their unlinked forms (e.g., in the absence of the association), are biologically active. The association between said residues is covalent or ionic and is either direct or indirect through a linker. The first small molecule can be the same or different from the second. The codrugs referred to herein may optionally be homocodrugs or heterocodrugs. A “homocodrug,” also termed a “symmetrical codrug,” refers to a codrug that produces, upon cleavage or dissociation, two or more molecules of a single drug, and no other drug molecules, i.e., the homocodrug is composed primarily of two or more residues of a single drug, without incorporating a residue of a second drug. A “heterocodrug,” also termed an “asymmetrical codrug,” refers to a codrug that produces, upon cleavage or dissociation, residues of at least two different drugs.

The term “prodrug” as used herein means a first small molecule residue associated with a second small molecule residue, wherein one of the residues is not biologically active. In some embodiments, the prodrug may be biologically inactive in its prodrug form. The association between said residues is covalent and can be either direct or indirect through a linker. Prodrugs of biologically active compounds include esters, as well as anhydrides, amides, and carbamates that are hydrolyzed hydrolyzed under physiological conditions to reveal the desired molecule. In other embodiments, the prodrug is converted by an enzymatic activity of the host animal.

On this basis, prodrug formulations are not generally classified as sustained release dosage forms. However, the ability to bioreversibly modify the physicochemical properties of a drug (to create a prodrug compound) allows for better pharmacokinetics or physiochemical properties and hence can influence the drug blood levels versus time profile of the drug. Thus, prodrug formulations can be used as a strategy for sustained release and sustaining therapeutic levels of pharmacological moieties in an individual. Thus, prodrug formulations can be used as a strategy for sustained release and sustaining therapeutic levels of pharmacological moieties in an individual.

The compound of the invention contains a first and second pharmacological moiety, and may also contain other pharmacological moieties (such as a third pharmacological moiety, and possibly a fourth pharmacological moiety, etc.). In one embodiment, the compound of the invention contains the first pharmacological moiety and the second pharmacological moiety in equimolar amounts. In a particular embodiment, the compound contains one first pharmacological moiety and one second pharmacological moiety.

The compound of the invention has several advantages for the treatment of hypertension. Among these are advantages for the patient, for the prescribing physician, for the surgeon and for the pharmacist (by reducing the number of active components in tablet formulation, each component having different properties). For the patient and the physician, the compound of the invention can enhance patient compliance by providing a convenient reduction in the number of pills to be taken. The compound of the invention can also be a drug compound that is superior to either pharmacological moiety, because the compound can have moieties with synergistic effects. The compound of the invention can also advantageously provide a pharmaceutical with improved bioavailability, since a single compound is administered. Moreover, any patient population variance can be assessed by the physician in terms of a single compound, rather than two compounds. With the compound of the invention, differences in absorption between the pharmacological moieties do not lead to different doses.

As used herein, the term “treating” or “treatment” includes reversing, reducing, or arresting the symptoms, clinical signs, and underlying pathology of a condition in manner to improve or stabilize a subject's condition. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

Covalent Linkages Between Pharmacological Moieties.

In one embodiment, the compounds of the invention are formed by covalent conjugation of two or more pharmacological moieties. See, EXAMPLES 1-2. Pharmacological moieties can be linked as a compound by reversible covalent bonds, such that at the desired site in the body, the covalently-linked pharmacological moieties are cleaved to regenerate the active forms of the pharmacological moieties, or the prodrug precursors to the drugs of interest. The rate of cleavage of the pharmacological moieties can be controlled by the type of the bond linking the pharmacological moieties, the choice of pharmacological moieties and the physical form of the compound.

The first and second pharmacological moieties may be covalently linked either by a direct covalent linkage or by an indirect covalent linkage, through a linker group (L group). This relationship can be generically expressed in the following Formula (I):
A₁-L-A₂ (I)
wherein A₁ and A₂ are the residues of the first pharmacological moiety and second pharmacological moiety, respectively, as defined above, and the linking (L) group is either a direct bond or a linker as described above. When the linking group is a direct bond, Formula (I) above may be expressed more compactly as Formula (II):
A₁A₂ (II)

When a compound of Formula I is exposed to physiologic fluids, such as blood plasma, it is subjected to hydrolysis.

In certain embodiments, compounds including L have a structure of formula (III): 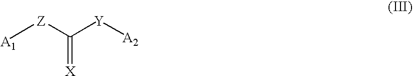
wherein Z is O, N, CH₂, CH₂O, or CH₂S;
Y is O or N; and
X is O or S.

Covalent linkages can be, for example, ester, carbonate, cyclic phosphate ester or carbamate bonds. The physiologically labile linkage may be any linkage that is labile under conditions approximating those found in physiologic fluids, such as blood plasma. The linkage may be a direct bond (for instance, an amide, carbonate, carbamate, sulfonate, or a sulfamate linkage) or may be a linking group (for instance, a C₁-C₁₂ dialcohol, a C₁-C₁₂ hydroxylalkanoic acid, a C₁-C₁₂ hydroxyalkylamine, a C₁-C₁₂ diacid, a C₁-C₁₂ amino acid, or a C₁-C₁₂ diamine). The linkage may be a direct amide, carbonate, carbamate, and sulfamate linkages, and linkages via succinic acid, salicylic acid, diglycolic acid, and halides thereof.

Preferred linkages are of the type —OC(O)CH₂—, —OC(O)O—, —OCH₂C(O)CH₂O—, —OCH₂CH(OH)CH₂O—, —OCH₂C(O)(OCH₂CH₂)ₙOC(O)CH₂O—, —O(CH₂)₃O—, —(OCH₂CH₂)ₙO—, 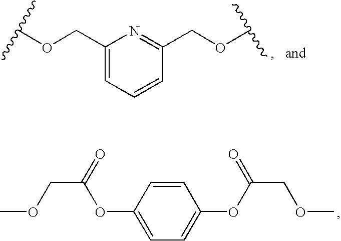
wherein n is an integer from 1 to 6.

The linkages can be labile under physiologic conditions, which is generally a pH of about 6 to about 8. The lability of the linkages depends upon the particular type of linkage, the precise pH and ionic strength of the physiologic fluid, and the presence or absence of enzymes that tend to catalyze hydrolysis reactions in vivo. In general, lability of the linkage in vivo is measured relative to the stability of the linkage when the compound has not been solubilized in a physiologic fluid. Thus, while some compounds of the invention may be relatively stable in some physiologic fluids, nonetheless, they are relatively vulnerable to hydrolysis in vivo (or in vitro, when dissolved in physiologic fluids, whether naturally occurring or simulated) as compared to when they are neat or dissolved in non-physiologic fluids (e.g. non-aqueous solvents such as acetone). Thus, the labile linkages are such that, when the drug is dissolved in an aqueous solution, especially a physiologic fluid such as blood plasma, the hydrolysis reaction lies heavily on the side of the hydrolysis products.

Moreover, the covalent bond can be enzyme-specific, for example, enzymatically labile to esterases, or may be designed to break down in specific areas, e.g., in the gastrointestinal tract, as it is crosses mucosa, or as it enters the blood stream. Alternatively, the covalent linkages can be chemically labile (e.g., base catalyzed hydrolysis of the linkage).

The first pharmacological moiety or second pharmacological moiety, or both, can be moieties that either possess, or may be adapted to possess, a group that may be condensed with a linkage to form a hydrolytically labile bond. Examples of such groups are hydroxy (—OH) groups, amine groups, acid (—COOH) groups, sulfonamide groups, and sulfonate (—SO₃H) groups.

The term “amide”, as used herein, refers to a group 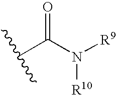
wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by 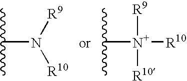
wherein R⁹, R¹⁰, and R¹⁰′ each independently represent a hydrogen or a hydrocarbyl group, or R⁹ and R¹⁰ taken together with the N atom to which they are attached complete a heterocycle having from 4 to 8 atoms in the ring structure.

The term “carbamate” is art-recognized and refers to a group 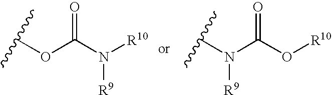
wherein R⁹ and R¹⁰ independently represent hydrogen or a hydrocarbyl group.

The term “carbonate” is art-recognized and refers to a group —OCO₂—.

The term “ester”, as used herein, refers to a group —C(O)OR⁹

wherein R⁹ represents a hydrocarbyl group.

The term “halide” as used herein means halogen and includes chloro, fluoro, bromo, and iodo.

The term “heterocycle” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The term “heterocycle” also includes polycyclic ring systems having two or more cyclic rings in which two or more carbons are common to two adjoining rings wherein at least one of the rings is heterocyclic. Heterocycle groups include, for example, piperidine, piperazine, pyrrolidine, morpholine, lactones, lactams, and the like.

The terms “hydrocarbyl” and “alkane” or “alkanoic”, as used herein, refers to a group that has is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a =O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The term “sulfamate” is art-recognized and refers to the group represented by the general formula

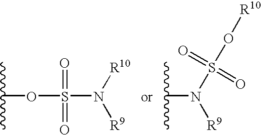

wherein R⁹ and R¹⁰ independently represents hydrogen or hydrocarbyl.

The term “sulfonamide” is art-recognized and refers to the group represented by the general formulae 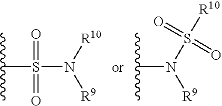
wherein R⁹ and R¹⁰ independently represents hydrogen or hydrocarbyl.

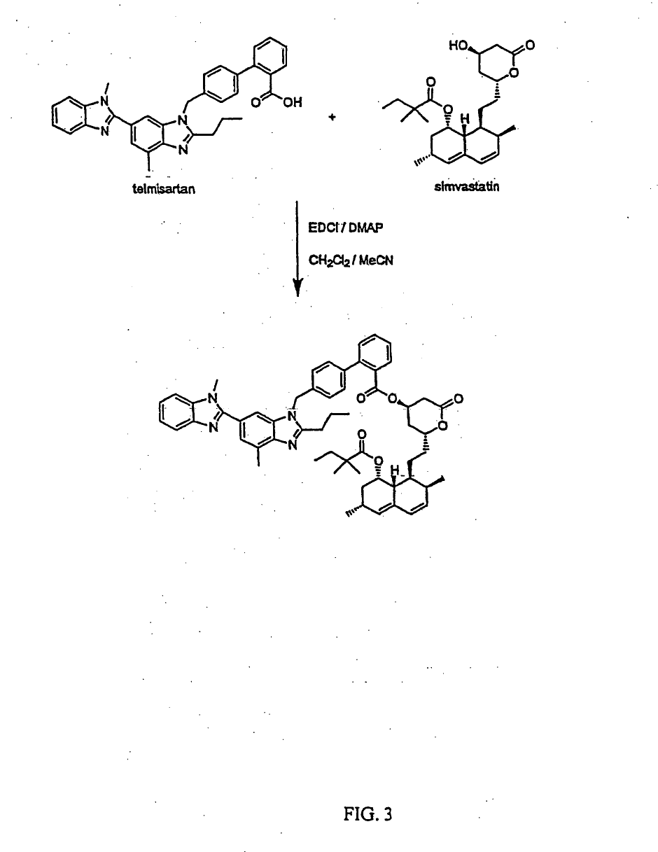

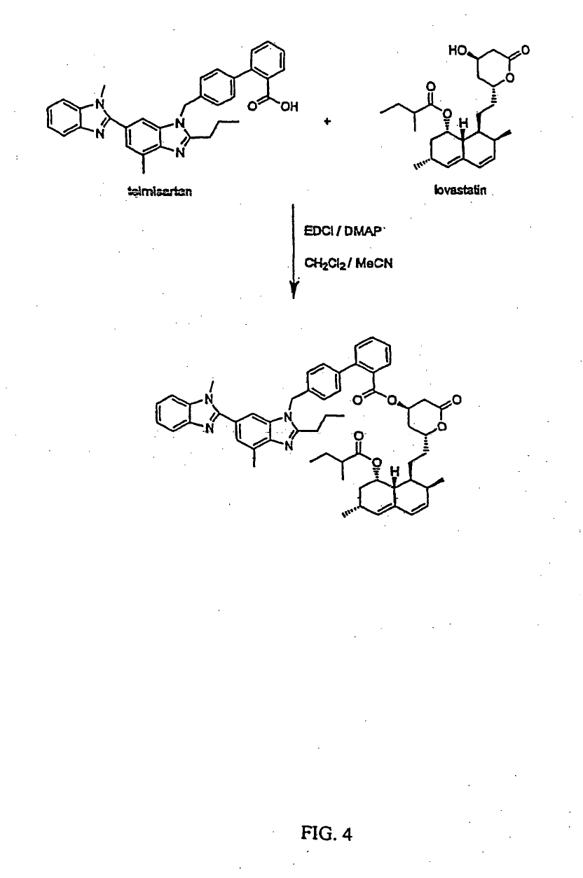

Exemplary reaction schemes for making the compounds of the invention are illustrated in FIGS. 3-4 (see particularly, EXAMPLES 1-2). These schemes can be generalized by substituting other statins for simvastatin or lovastatin. Likewise, these schemes can be generalized by substituting other angiotensin receptor blockers for telmisartan. These schemes can also be generalized by using appropriate linkers and agents as starting materials.

In general, where the first pharmacological moiety and the second pharmacological moiety are to be directly linked, the first pharmacological moiety is condensed with the second pharmacological moiety under conditions suitable for forming a linkage that is labile under physiologic conditions. In some cases, it is necessary to block some reactive groups on one, the other, or both of the moieties.

Where the moieties are to be covalently linked via a linker, such as succinic acid or diglycolic acid, the first pharmacological moiety can initially be condensed with the linker. Sometimes, it is advantageous to perform the reaction in a suitable solvent, such as acetonitrile, in the presence of suitable catalysts, such as carbodiimide and dimethylaminopyridine (DMAP), a nucleophilic catalyst, or under conditions suitable to drive off water of condensation or other reaction products (e.g. reflux), or a combination of two or more thereof. After the first moiety is condensed with the linker, the combined first moiety and linker may then be condensed with the second pharmacological moiety. Again, in some cases, it is advantageous to perform the reaction in a suitable solvent, such as acetonitrile, in the presence of suitable catalysts, or under conditions suitable to drive off water of condensation or other reaction products (e.g. reflux), or a combination of two or more thereof. Where one or more active groups have been blocked, it may be advantageous to remove the blocking groups under selective conditions, however it may also be advantageous, where the hydrolysis product of the blocking group and the blocked group is physiologically benign, to leave the active groups blocked.

The active groups can be derivatized to increase their reactivity. For instance, where the first moiety is an acid and the second moiety is an alcohol (i.e. has a free hydroxyl group), the first moiety may be derivatized to form the corresponding acid halide, such as an acid chloride or an acid bromide. Other possibilities are known in the art for increasing yield, lowering production costs, improving purity, etc. of the compound of the invention by using conventionally derivatized starting materials to make compounds of the invention.

While diacids, dialcohols, amino acids, etc. are described above as being suitable linkers, other linkers are also within the scope invention. For example, while the hydrolysis product of a compound of the invention may comprise a diacid, the actual reagent used to make the linkage may be, for example, a diacetylhalide, such as a diacetylchloride or diacetylbromide, or a dianhydride. Other possible acid, alcohol, amino, sulfate, and sulfamoyl derivatives may be used as reagents to make the corresponding linkage.

In one advantageous embodiment of the invention, codrugs can be used to deliver the active metabolite to the person being treated. A prodrug may have no pharmacologic activity until metabolically converted into an active compound. When the metabolite of a drug produces the therapeutic effect, it is considered an “active metabolite”. For example, the first pharmacological moiety can be a hydroxy acid having structure similar to the product lactone hydrolysis of an HMGCoA reductase inhibitor, such as lovastatin, simvastatin, atorvastatin, or cerivastatin (but not pravastatin or fluvastatin). For the first phar mnacological moiety, the open-ring hydroxy acid is often the active metabolite.

Ionic Linkages Between Pharmacological Moieties.

In one embodiment, the compounds are formed by ionic interactions between two or more pharmacological moieties. See, U.S. Pat. No. 6,051,576, which is incorporated herein by reference.

Salt formation is an acid-base reaction involving either a proton-transfer or neutralization reaction and is therefore controlled by factors influencing such reactions. Theoretically, every compound that exhibits the appropriate acid or base characteristics can participate in salt formation. Particularly important is the relative strength of the acid or base and the acidity and basicity constants of the pharmacological moieties involved. These factors determine whether or not salt formation occurs and are a measure of the stability of the resulting salt. The salt form is known to influence a number of physico-chemical properties of the parent compound including dissolution rate, solubility, stability, and hygroscopicity. Salt formation is useful in pharmaceutical formulations since these properties, in turn, affect the availability and formulation characteristics of the drug.

In one exemplary procedure to make the compound of the invention, the first pharmacological moiety is dissolved in an organic solvent together with an equivalent amount of the second pharmacological moiety. The solution is then evaporated under a nitrogen atmosphere at room temperature to a liquid/semi-solid viscous mass. The compound is then crystallized through the use of a suitable organic solvent such as alcohol, etc. The remainder of the liquid can be driven off through the continued application of heat. The compound is then formulated into any one of a number of known dosage forms or delivery systems by means known in the art. See, U.S. Pat. No. 5,385,941. See also, published PCT applications WO 99/11259 and WO 00/73298. Other suitable procedures for forming such salts will be well known to those of skill in the art.

Moreover, the compound of the invention can be or can be formulated as a mineral acid salt, a carboxylic acid salt, or an amino acid salt.

In certain embodiments, the first pharmacological moiety is an HMGCoA reductase inhibitor. HMGCoA reductase inhibitors (also known as statins) are currently the most effective drugs in the battle against high cholesterol. Additionally, statins have been found to have beneficial activity in the treatment or inhibition of inflammation and multiple sclerosis, and the treatment or prophylaxis of Alzheimer's disease, diabetes, osteoporosis, and stroke. Accordingly, compounds that comprise at least one HMGCoA reductase inhibitor (or preferably two, such as a dimer or heterodimer) may be used to treat or inhibit inflammation and/or multiple sclerosis. In another embodiment, compounds that comprise at least one HMGCoA reductase inhibitor (or preferably two, such as a dimer or heterodimer) may be used in the treatment or prophylaxis of Alzheimer's disease and/or diabetes and/or osteoporosis and/or stroke.

The regulation of cholesterol biosynthesis has long been the subject of intensive research because of its connection with atherosclerosis, cerebrovascular and coronary cardiovascular disease. Control of cholesterol synthesis occurs mainly at the first committed step in the pathway, catalyzed by 3-hydroxy-3-methylglutaryl CoA (HMGCoA) reductase. Statins block hydroxymethylglutaryl-CoA reductase (EC 1.1.1.34), an enzyme needed in the formation of cholesterol. Other names for the enzyme include hydroxymethylglutaryl coenzyme A reductase (reduced nicotinamide adenine dinucleotide phosphate); 3-hydroxy-3-methylglutaryl-CoA reductase; β-hydroxy-β-methylglutaryl coenzyme A reductase; hydroxymethylglutaryl CoA reductase (NADPH); S-3-hydroxy-3-methylglutaryl-CoA reductase; NADPH-hydroxymethylglutaryl-CoA reductase; HMGCoA reductase-mevalonate:NADP-oxidoreductase (acetylating-CoA); 3-hydroxy-3-methylglutaryl CoA reductase (NADPH) and (R)-mevalonate:NADP oxidoreductase (CoA-acylating). The enzyme catalyzes the conversion of (S)-3-hydroxy-3-methylglutaryl-CoA+2 NADPH₂ to (R)-mevalonate+CoA +2 NADP.

Among the statin class of drugs are Lipitor® (atorvastatin); Pravachol® (pravastatin); Zocor® (simvastatin); Mevacor® (lovastatin); Lescol® (fluvastatin); Baycol® (cerivastatin), Crestor® (rosuvastatin), mevastatin, pitavastatin, dalvastatin, glenvastatin, dihydromevinolin, SDZ-265859, BMS-180431, CP-83101, and L-669262.

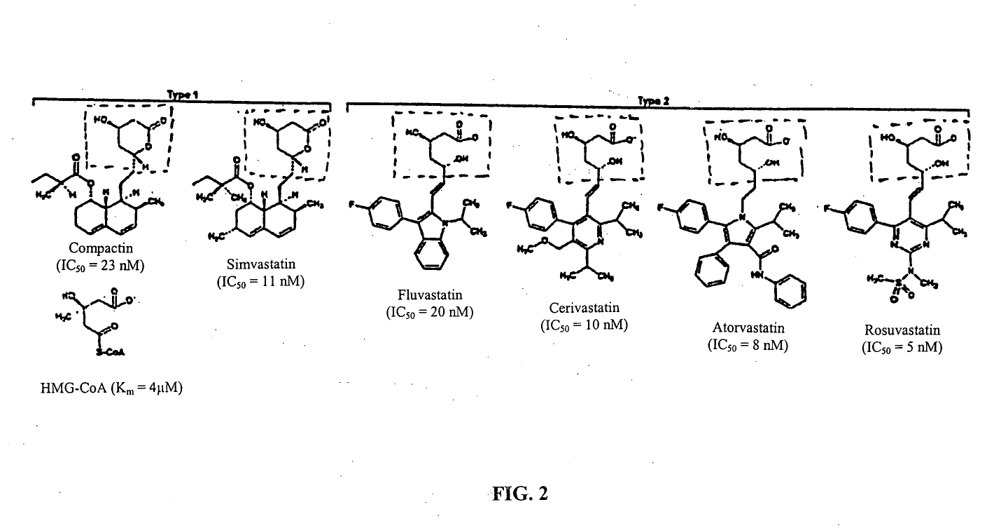

The structure of the statin class of compounds is known to those of skill in the pharmacological arts. Statins generally are known in the art to share an HMG-like moiety (see, FIG. 2). The statins generally are known to share rigid, hydrophobic groups that are covalently linked to the HMGCoA-like moiety. Lovastatin, pravastatin, and simvastatin resemble the substituted decalin-ring structure of Compactin (also known as mevastatin). Istvan, E. S. & Deisenhofer, J., Science 292: 1160-64 (2001) classify this group of inhibitors as type 1 statins. Fluvastatin, cerivastatin, atorvastatin, and rosuvastatin are fully synthetic HMGCoA reductase inhibitors with larger groups linked to the HMG-like moiety. Istvan & Deisenhofer refer to these inhibitors as type 2 statins. The additional groups range in character from very hydrophobic (e.g., cerivastatin) to partly hydrophobic (e.g., rosuvastatin). All statins are competitive inhibitors of HMGR with respect to binding of the substrate HMGCoA, but not with respect to binding of NADPH. The Kᵢ (inhibition constant) values for the statin-enzyme complexes range between 0.1 to 2.3 nM, whereas the Michaelis constant, Kₘ, for HMGCoA is 4 μM.

Istvan & Deisenhofer have determined how the structures of the catalytic portion of human HMGCoA reductase are complexed with different statins. The bulky, hydrophobic compounds of statins occupy the HMG-binding pocket and part of the binding surface for CoA. Thus, access of the substrate HMGCoA to HMGCoA reductase is blocked when statins are bound.

Statins have proven to be very effective at lowering blood cholesterol levels and also at preventing heart attacks, which is one of the main consequences of high cholesterol levels. The process by which cholesterol causes the damage is known as atherosclerosis and involves the build-up of cholesterol-containing plaques in the walls of the arteries, which can eventually block them altogether. The plaque in the arteries supplying the heart results in a heart attack, and in the arteries supplying the brain, causes stroke.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount. Prevention of an infection includes, for example, reducing the number of diagnoses of the infection in a treated population versus an untreated control population, and/or delaying the onset of symptoms of the infection in a treated population versus an untreated control population. Prevention of pain includes, for example, reducing the magnitude of, or alternatively delaying, pain sensations experienced by subjects in a treated population versus an untreated control population.

Statins generally have few side effects, and help not only to lower overall cholesterol, LDL (so-called “bad”) cholesterol and triglycerides, but also to increase HDL (so-called “good”) cholesterol. Primary and secondary prevention trials have shown that use of statins to lower an elevated low-density lipoprotein cholesterol level can substantially reduce coronary events and death from coronary heart disease. Strong evidence in support of lipid lowering as a means of secondary coronary heart disease prevention comes from three large trials, the Scandinavian Simvastatin Survival Study (4S study) (Lancet 344:1383-9 (1994)), Long-Term Intervention with Pravastatin in Ischaemic Disease (LIPID) study (Sacks F M, et al., N. Engl. J. Med. 335:1001-9 (1996)) and the Cholesterol and Recurrent Events (CARE) trial (N. Engl. J. Med. 339:1349-57 (1998)) in which treatment with HMGCoA reductase inhibitors (statins) reduced coronary events and reduced mortality. Studies have also shown that some statins are effective in preventing not only recurrent heart attacks, but first heart attacks as well. Some statins are also effective in reducing the risk of strokes. New studies have shown that even people with ordinary cholesterol levels might benefit from taking cholesterol-lowering drugs. Statin therapy is indicated for primary prevention in hypertensive subjects up to 70 years old with a cholesterol level of greater than 5 mmol/L and a 10-year coronary artery disease risk of greater than 30%. Ramsay, L. E., J. Human Hypertension 13: 569-592 (1999) and Ramsay, L. E., British Med. J. 319: 630-635 (1999).

Unfortunately, statins are under-prescribed. The National Cholesterol Education Program has promulgated guidelines for cholesterol screening and treatment (Arch. Intern. Med. 148:36-69 (1988) and National Cholesterol Education Program, NIH publication no. 97-3794 (1997)). Thus far, however, primary care physicians have inadequately adopted these guidelines in clinical practice (see, Am. Fam. Physician 63: 309-20, 323-4 (2001)). Moreover, even when prescribed, patient compliance is a problem.

Some typical daily dosages for oral administration of statins are shown in the TABLE 1 below:

TABLE 1

| Statin | Usual daily dose range | |
|---|---|---|
| Lovastatin | 10-80 | mg/day |
| Simvastatin | 5-40 | mg/day |
| Pravastatin | 10-80 | mg/day |
| Fluvastatin | 20-40 | mg/day |

In certain embodiments where the first pharmacological moiety is a HMGCoA reductase inhibitor, the second pharmacological moiety is selected from angiotensin receptor blockers, cholesterol absorption inhibitors, cholesteryl ester transfer protein inhibitor, and HMGCoA reductase inhibitors.

Angiotensin Receptor Blocker (ARB).

Angiotensin receptor blockers have specific effects on the systems that are affected by angiotensin II that is different from other classes of pharmacological agents, such as angiotensin converting enzyme (ACE) inhibitors. Because of this differential effect, angiotensin receptor blockers are better tolerated by patients, as is evident in lower side-effect profiles with angiotensin receptor blockers (see, Annals of Long-Term Care 7[8]: 305-308 (1999))

The final active messenger of the renin-angiotensin pathway is angiotensin II. Angiotensin II elevates blood pressure by a variety of mechanisms, including direct vasoconstriction, potentiation of sympathetic nervous system activity at both central and peripheral levels, stimulation of aldosterone synthesis and release with consequent sodium and fluid retention by the kidney and stimulation of arginine vasopressin release. In addition, angiotensin II has a variety of actions that damage blood vessels directly. Angiotensin II also plays a role in the vascular injury response, stimulating leukocyte adhesion to the site of injury and favoring superoxide and peroxynitrite formation and proliferation and migration of various cell types toward the luminal site of injury, which events result in atherosclerotic plaque or fibrous neointima formation. Angiotensin II and some of its constituent peptides also stimulate synthesis of the antithrombolytic agent, PAI-1, suggesting that activation of the renin-angiotensin-aldosterone system predisposes to atherosclerosis and thromboembolic events, including heart attack and stroke.

Angiotensin II binds to AT₁ receptors to cause vasoconstriction and fluid retention, both of which lead to an increase in blood pressure. The angiotensin receptor blockers lower blood pressure by blocking the AT₁ receptors, one of four receptors with which angiotensin II can interact to cause changes in the cell. Brown, N. J. & Vaughn D. E., Circulation 97:1411-1420 (1998). AT₁ receptor blockers block the intrinsic signaling of the AT₁ receptor, thus offering a more complete blockade of angiotensin II than other anti-hypertensive pharmacological agents, and potentially, greater protection against myocardial damage.

AT₁ receptor blockers are nonpeptide analogues of aniogiotensin II. Burnier M. & Brunner H. R., Lancet 355: 637-45 (2000). Drugs from the angiotensin receptor blockers class include candesartan (candesartan cilexetil; Atacand®; Blopress®), irbesartan (Avapro®), losartan (Cozaar®), telmisartan (Micardis®), valsartan (Diovan®), and eprosartan (Teveten®). The suffix “-sartan” distinguishes this class from other anti-hypertensive pharmacological agents. Angiotensin receptor blockers differ somewhat in their chemical structure, potency, bioavailability, plasma half-life (telmisartan has the longest half-life; losartan, the shortest), and metabolism. Angiotensin receptor blockers do not act as prodrugs (e.g., inactive until converted by the liver to active agents). Losartan, however, has an active metabolite that also serves to extend the duration of drug action.

The angiotensin receptor blockers are generally taken once a day and do not commonly produce significant side effects. However, clinical practice suggests that some agents should be used twice a day to achieve adequate blood pressure goals as outlined by The Sixth Report of the Joint National Committee on the Prevention, Detection, Evaluation, and Treatment of High Blood Pressure. Arch. Intern. Med. 157 (1997). Rarely, they interfere with or worsen kidney function.

Angiotensin receptor blockers differ in how they are handled in the human body.

TABLE 2

| Angiotensin | Active | Major route of | Half-life | Usual daily dose |
|---|---|---|---|---|
| Blockers | metabolite | inactivation | (hr) | range (mg) |
| Losartan | Yes | Liver | 5 | 25-100 |
| Valsartan | No | Liver | 6 | 80-160 |
| Irbesartan | No | Liver | 13 | 75-300 |
| Candesartan | Yes | Liver and renal | 10 | 4-16 |

A large-scale trial has evaluated the effects of an angiotensin receptor blocker in elderly patients with heart failure, the Evaluation of Losartan in the Elderly (ELITE) trial. Pitt, B. et al., Lancet 349:747-752 (1997). Overall, the results showed that the angiotensin receptor blocker drug was better tolerated than the ACE inhibitor, specifically as related to the areas of renal function, hyperkalemia, and cough.

Cholesterol Absorption Inhibitor.

Cholesterol absorption inhibitors selectively inhibit the absorption of cholesterol and related phytosterols in the small intestine. This class of inhibitor does not inhibit cholesterol synthesis in the liver, but rather localizes and appears to act at the brush border of the small intestine and inhibit the absorption of cholesterol, leading to a decrease in the delivery of intestinal cholesterol to the liver. This causes a reduction of hepatic cholesterol stores and an increase in clearance of cholesterol from the blood. This distinct mechanism of action is complementary to that of HMGCoA reductase inhibitors. Ezetimibe (Zetia™) is the first member of this class of drugs to be approved by the FDA, and has been found to reduce total cholesterol, low density lipoprotein cholesterol (LDL), triglycerides (TG), and apolipoprotein (Apo B), the major protein constituent of LDL. Additionally, ezetimibe has been shown to raise high density lipoprotein cholesterol (HDL) in patients with hypercholesterolemia.

Additionally, it has been shown that concurrent administration of ezetimibe with an HMGCoA reductase inhibitor selected from atorvastatin, simvastatin, pravastatin, and lovastatin, results in significantly lowered total cholesterol, LDL, Apo B, and TG, and, with the exception of pravastatin, increased HDL compared to the HMGCoA reductase inhibitor administered alone. The clinical studies also demonstrated that when administered alone, ezetimibe reduces LDL by 17%, whereas when ezetimibe is administered in combination with an either simvastatin or atorvastatin, LDL was lowered by an additional 12 to 20%. The typical dosage and administration of ezetimibe is 10 mg once daily either alone or in combination with an HMGCoA reductase inhibitor.

Cholesteryl Ester Transfer Protein Inhibitor.

The second pharmacological moiety may be an agent that inhibits cholesteryl ester transfer protein and increases HDL cholesterol levels such as torcetrapib or an active metabolite thereof.

HMGCoA Reductase Inhibitor.

In certain embodiments, the second pharmacological moiety may be an HMGCoA reductase inhibitor, either the same or different from the first HMGCoA reductase inhibitor. Compounds of the present invention comprising two HMGCoA reductase inhibitors preferably have improved properties as compared to properties of the separate compounds from which they are derived.

For example, the first HMGCoA reductase inhibitor may decompose more slowly under ambient conditions and/or ordinary storage conditions (and thereby have a longer shelf life) when linked to a second HMGCoA reductase inhibitor, as compared to an unlinked HMGCoA reductase inhibitor. In another aspect, linking two HMGCoA reductase inhibitors together may provide for easier formulation as compared to the formulation of its unlinked constituent compounds. For example, the linked compound may be more soluble in a polymeric delivery system. In some embodiments, linking two HMGCoA reductase inhibitors may provide a compound that is more readily mixed with a pharmaceutically acceptable carrier. In still other embodiments, linked HMGCoA reductase inhibitors may more readily be adapted than the unlinked constituent compounds for use in solid dosage forms, e.g., where the linked HMGCoA reductase inhibitors are a solid at room temperature and one or more unlinked constituent compounds are liquids at room temperature. In such embodiments, the constituent compounds may be prepared, stored, and/or delivered with greater convenience and/or efficiency in when linked together than in the unlinked form.

The invention also relates to compounds comprising a first pharmacological moiety connected to at least a second pharmacological moiety through a physiologically labile linker, or a salt thereof, wherein both pharmacological moieties, when active or when activated, act to reduce cardiovascular disease. The pharmacological moieties are each independently selected from ACE inhibitors, cardioprotective agents, steroids and corticosteroids, sex steroids, apoptosis inhibitors, agents that alter expressions/activity of MMPs, and anti-inflammatory agents. The two or more pharmacological moieties can be linked either by covalent bonds or by ionic interactions.

ACE Inhibitors.

The second pharmacological moiety may be an ACE inhibitor. Suitable ACE inhibitors include, but are not limited to, captopril, zofenopril, fosinopril, enalapril, enamapril, ceranopril, cilazopril, delapril, pentopril, quinapril, ramipril, lisinopril, and salts of such compounds.

Cardioprotective Agents.

The second pharmacological moiety may be a cardioprotective agent. Suitable cardioprotective agents include, but are not limited to, verapamil, diltiazem, digitalis, and adenosine.

Steroids and Corticosteroids.

The second pharmacological moiety may be a steroid or corticosteroid. Suitable steroids include, but are not limited to acetoxypregnenolone, al dlometasone, aldosterone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylaminoacetate, prednisolone sodium phosphate, prednisone, prednival, prednylidene, rimexo lone, tixocortol, triamcinolone, triamcinolone acetonide, triamcinolone benetonide, and triamcinolone hexacetonide. In a preferred embodiment, the steroidal antiinflammatory agent is selected from the group consisting of cortisone, dexamethasone, hydrocortisone, methylprednisolone, prednisolone, prednisone, and triamcinolone, and derivatives thereof such as acetonides and lower alkanoate esters such as acetates, propionates, and butyrates.

Sex Steroids.

The second pharmacological moiety may be a sex steroid. Suitable sex steroids include, but are not limited to, androgens (such as testosterone, androstenedione, dihydrotestosterone, and dehydroepiandrosterone), estrogens (such as estradiol and diethylstilbestrol), and progestagens (such as progesterone and progestins).

Apoptosis Inhibitors.

The second pharmacological moiety may be an apoptosis inhibitor. Apoptosis inhibitors are a class of agents including, but not limited to Bax, Bik/Nbk, Bak, Bad, and Bid [See Peter, et al., Proc. Nat. Acad. Sci. 94:12736-12737 (1997) which is incorporated herein in its entirety].

Agents that Alter Expressions/Activity of MMPs.

The second pharmacological moiety may be an MMP inhibitor. Suitable MMP inhibitors include, but are not limited to, 4-[4-(4-fluorophenoxy)benzenesulfonylamino]tetrahydropyran-4-carboxylic acid hydroxyamide; 5-Methyl-5-(4-(4′-fluorophenoxy)-phenoxy)-pyrimidine-2,4,6-trione; 5-n-Butyl-5-(4-(4′-fluorophenoxy)-phenoxy)-pyrimidine-2,4,6-trione and prinomistat.

Anti-Inflammatory Agents.

The second pharmacological moiety may be an anti-inflammatory agent. Suitable anti-inflammatory agents include, but are not limited to diclofenac, etoldolac, fenoprofen, floctafenine, flurbiprofen, ibuprofen, indoprofen, ketoprofen, ketorolac, lomoxicam, morazone, naproxen, perisoxal, pirprofen, pranoprofen, suprofen, suxibuzone, tropesin, ximoprofen, zaltoprofen, zileuton, and zomepirac, and pharmaceutically acceptable salts, esters, prodrugs and protected forms thereof.

Diagnosis of Hypertension.

Diagnosis of hypertension and hypertension-related conditions, and the identification of individuals who would benefit by medical treatment for hypertension, are standard medical diagnoses. Further guidance may be obtained from The International Society of Hypertension and the World Health Organization (J. Hypertension 17: 151-183 (1999)), which suggest that young, middle-aged or diabetic subjects should be treated to a target blood pressure less than 130/80 mm Hg and the elderly to less than 140/90 mm Hg. The British Hypertension Society guidelines recommend the initiation of treatment with a systolic blood pressure greater than or equal to 160 mm Hg or a diastolic blood pressure greater than or equal to 100 mm Hg. The British Hypertension Society suggests that subjects with a blood pressure between 140-159 mm Hg systolic and 90-99 mm Hg diastolic should be treated in the presence of other risk factors, aiming for a target blood pressure less than 140/85 mm Hg. In diabetic patients the British Hypertension Society aim is to reduce blood pressure to less than 140/80 mm Hg. Other guidance is provided in TABLE 3.

TABLE 3
Diagnostic Classification for Hypertension by JNC-V (1993)

| Category | Systolic(mm HG) | Diastolic(mm Hg) |
|---|---|---|
| Normal | <130 | <85 |
| High normal | 130-139 | 85-89 |

Hypertension

| | | |
|---|---|---|
| Stage 1 | 140-159 | 90-99 |
| Stage 2 | 160-179 | 100-109 |
| Stage 3 | 180-209 | 110-119 |
| Stage 4 | >210 | >120 |

Dosages and Formulations for Oral Administration.

Dosages for administration of the compounds of the invention may be calculated by those of skill in the art (see, Goodman & Gilman, The Pharmacological Basis of Therapeutics, 8th Ed. (Pergamon Press, NY 1990); and The Merck Index, 11th Ed. (Merck and Co., Inc., Rahway, N.J. 1989); both incorporated herein by reference). Dosages are preferably in the range of about 1 to about 500 mg/kg body weight, and are administered preferably 1 to 2 times a day. Additional guidance for the appropriate dosage for oral administration of compounds may be found in the dosages for the first pharmacological moiety and second pharmacological moiety, respectively. See, TABLES 1 and 2. From published studies of administration of the first pharmacological moiety and second pharmacological moiety and the information known to those of skill in the art, appropriate therapeutic ranges for administration of the compounds of the invention can reasonably be estimated. As one example, from the information provided in TABLES 1 and 2, the compound of the invention can be administered with a range of effective dosages. The lower end of the range can be 1 μg/day, more particularly 1 mg/day, more particularly 5 mg/day, more particularly 10 mg/day, more particularly 20 mg/day, more particularly 25 mg/day, more particularly 75 mg/day, or 80 mg/day. The upper end of the range can be 300 mg/day, more particularly 160 mg/day, more particularly 100 mg/day, more particularly 80 mg/day, or 40 mg/day. The compound of the invention is administered only once or at most twice a day.

The compounds of the invention are labile when dissolved in bodily fluids and are rapidly hydrolyzed to regenerate the two active parent drugs. In the solid form, however, they are stable, even in an aqueous environment because in order to hydrolyze they must first be in solution.

Other Dosages and Formulations.

The method of the invention advantageously employs a compound of the invention, which may be delivered to an individual in need thereof in an art recognized manner, such as via intravenous, subcutaneous, intramuscular or other parenteral mode of injection, or by surgical implantation. Although intravenous injection is possible, the properties of the compounds of the invention make them well-suited for subcutaneous or intramuscular implantation or injection into soft tissue.

Compounds of the invention can also be formulated as suspensions (nanoparticle size range) and upper size limitations are only imposed the application method under consideration.

In an embodiment of the invention, a compound of the invention is prepared in a solid form, such as a pellet that may be directly injected. Pellets of a compound of the invention can slowly release drugs in solution or into bodily fluids, reflecting the low solubility of the conjugated forms. Pellets may be formulated from the compounds alone or with implantable, bioerodible substances such as polylactic acid and polyglycolic compounds. Pellets may be formulated by methods known in the art and may contain 0.1 to about 100% of the composition.

In other embodiments of the invention, the compound of the invention is prepared in an anhydrous solution or suspension, for instance in vegetable oil, such as palm oil, and injected intramuscularly. The compound of the invention may be administered in injectable form such as in liposomes, liquids, suspensions, microspheres or nanoparticles. Preparation of such aqueous solutions, liposomes, emulsion and suspensions are known to those of ordinary skill in the art (see, Remington's Pharmaceutical Sciences, 18th Ed. (Mack Publishing Co., Easton, Pa., 1990)).

In another embodiment, the compound is an oral formulation, such as in capsules, tablets, or gelcaps. In yet another embodiments of the invention, the compound is in a topically applicable form, such as a transdermal patch, ointment, cream, suspension, liquid, elixir or eye drop (see, Remington's Pharmaceutical Sciences, 18th Ed. (Mack Publishing Co., Easton, Pa., 1990)).

Controlled Delivery Systems.

In one embodiment of the invention, compounds of the invention (either solid, liquid or colloidal) are contained in controlled delivery systems for a controlled or sustained release of compounds for a systemic or local pharmacological or physiological effect relating to hypertension and hypertension-related disease states. Such disease states are known to those of ordinary skill in the art (see, Goodman & Gilman, The Pharmacological Basis of Therapeutics, 8th Ed. (Pergamon Press, NY, 1990); and The Merck Index, 11th Ed. (Merck and Co., Inc., Rahway, N.J. 1989); both incorporated herein by reference).

The controlled delivery system is preferably chosen such that the compound has a rate of diffusion from the polymer matrix under physiologic conditions be not rate-limited by the permeability of the polymer matrix. See, U.S. Pat. No. 6,051,576, incorporated by reference, for a discussion of controlled delivery systems.

Formulations of the compounds of the invention may also contain several other substituents to optimize release, bioavailability or appearance and may be used in sustained release devices or systems. Such substituents are known to those of ordinary skill in the art and, for example, are set forth in Remington's Pharmaceutical Sciences, 18th Ed. (Mack Publishing Co., Easton, Pa., 1990). Furthermore, the compounds may be conjugated to another agent to reduce the undesirable effects such as isoniazid with pyroxidine. Another embodiment of the invention is a compound formulated with other drug or prodrug molecules.

A compound of the invention may also be formulated in bioerodible or nonbioerodible delivery systems to further control their release. Such bioerodible systems may include polylactic acid (bioerodible) to form a film around, or a matrix with a compound to further improve the pharmaceutical properties. Polylactic acid can be formulated in solutions of 2, 5 and 10% polylactic acid, and has been used to produce pellets attached to sutures. A totally bioerodible sustained release system for pharmacologically active agents may be composed of a compound of the invention in a formulation with another bioerodible substance such as polyvinyl acid, polyanyhydride, collagen, or polyalkylcyanoacrylates such as polybutylcyanoacrylate. 2% polyvinyl alcohol has been used to coat pellets of for subconjunctival delivery. Polybutyl cyanoacrylate (bioerodible) has also been used to form a matrix with pellets.

In another embodiment of the invention, compounds of the invention are contained in a nonerodible matrix or reservoir system containing natural or synthetic polymers that are biologically compatible with and essentially insoluble in body fluids. Such materials include for example, but are not limited to polyvinyl acetate, polyvinyl alcohol, cross-linked polyvinyl butyrate, ethylene ethyl acrylate copolymer, polyethyl hexyl acrylate, polyvinyl chloride, polyvinyl acetals, plasticized ethylene vinyl acetate copolymer, ethylene vinyl chloride copolymer, polyvinyl esters, polyvinyl butyrate, polyvinyl formal, polyamides, polymethyl methacrylate, polybutyl methacrylate, plasticized polyvinyl chloride, plasticized nylon, plasticized soft nylon, plasticized polyethylene terethphalate, natural rubber, polyisoprene, polyisobutylene, polybutadiene, polyethylene, polytetrafluoroethylene, polyvinylidine, chloride, polyacrylonitrile, cross-linked polyvinyl pyrrolidone, polytrifluorochloroethylene, chlorinated polyethylene, poly(1,4-isopropylidne diphenylene carbonate), vinylidine chloride, acrylonitrile copolymer, vinyl chloride-diethyl fumarate copolymer, silicone rubbers (especially medical grade polydimethylsiloxanes, ethylene-propylene rubber, silicone-carbonate copolymers, vinylidine chloride-vinyl chloride copolymer, vinyl chloride-acrylonitrile copolymer and vinylidine chloride acrylonitrile copolymer.

Systems containing the compounds of the invention may be directly implanted in a site in the vicinity of the surgical incision, in the vicinity of soft tissues, or both. In some embodiments of the invention, it may be desirable to combine a compound of the invention with one or more polymer vehicles. Such polymer vehicle may be any physiologically tolerated polymer, such as a bioerodible or a non-bioerodible polymer.

A polymer useful in a composition of the invention includes any biologically tolerated polymer that is permeable to a compound of the invention or that is bioerodible so that it releases the compound of the invention in a sustained-release manner. In preferred embodiments of the invention, the polymer has a permeability such that the permeability is not the principal rate determining factor in the rate of release of the compound of the invention from the polymer. In some embodiments of the invention, the polymer is non-bioerodible. Examples of nonbioerodible polymers useful in the invention include polyvinyl alcohol and polyurethane. In other embodiments of the invention, the polymer is bioerodible. Examples of bioerodible polymers useful in the invention include polyanhydride, polylactic acid, polyglycolic acid, polyorthoester, polyalkylcyanoacrylate or derivatives and copolymers thereof. Those of skill in the art will recognize that the choice of bioerodibility or nonbioerodibility of the polymer depends upon the final physical form of the system, as described in greater detail below. Other exemplary polymers include polysilicone and polymers derived from hyaluronic acid. The skilled artisan will understand that the polymer is prepared under conditions suitable to impart permeability such that it is not the principal rate determining factor in the release of the low solubility agent from the polymer.

Moreover, suitable polymers include naturally occurring materials (such as collagen or hyaluronic acid) or synthetic materials that are biologically compatible with bodily fluids and mammalian tissues, and essentially insoluble in bodily fluids with which the polymer will come in contact. In addition, the suitable polymers essentially prevent interaction between the low solubility agent dispersed/suspended in the polymer and proteinaceous components in the bodily fluid. The use of rapidly dissolving polymers or polymers highly soluble in bodily fluid or which permit interaction between the low solubility agent and proteinaceous components are to be avoided since dissolution of the polymer or interaction with proteinaceous components would affect the constancy of drug release.

Other suitable polymers include polypropylene, polyester, polyethylene vinyl acetate (PVA), polyethylene oxide (PEO), polypropylene oxide, polycarboxylic acids, polyalkylacrylates, cellulose ethers, polyalkyl-alkyacrylate copolymers, polyester-polyurethane block copolymers, polyether-polyurethane block copolymers, polydioxanone, poly-(β-hydroxybutyrate), polylactic acid (PLA), polycaprolactone, polyglycolic acid, and PEO-PLA copolymers.

Further suitable polymers are set forth in U.S. Pat. No. 6,051,576, incorporated herein by reference.

The details of one or more embodiments of the invention are set forth in the accompanying description above. Although any methods and materials similar or equivalent to those described herein can be used in the practice or testing of the invention, the preferred methods and materials are now described. Other features and advantages of the invention will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this invention belongs. All patents and publications cited in this specification are incorporated by reference.

### EXAMPLES

The following examples are presented in order to more fully illustrate the preferred embodiments of the invention. These examples should in no way be construed as limiting the scope of the invention, as defined by the appended claims.

### Example 1

### Telmisartan with Simvastin (FIG. 3)

Telmisartan (51 mg), EDCI (24 mg) and catalytic amount of DMAP were dissolved in 1.5 ml of dichloromethane and 0.5 ml of acetonitrile at 0-5° C. under argon. After 15 min., simvastatin (44 mg) was added and the resulting solution was stirred in an ice bath for 15 min. and then at room temperature overnight. The reaction mixture was diluted with dichloromethane and washed subsequently with sodium bicarbonate aq., water, brine and dried over anhydrous sodium sulfate. Evaporation of the solvent afforded colorless oil, which was purified by preparative thin layer chromatography (TLC). Chromatographic purification yielded 22 mg of the pure product.

¹H-NMR (CDCl₃), 0.70 (t, 3H), 0.82 (m, 6H), 1.10 (m, 9H), 2.78 (s, 3H), 3.02 (m, 2H), 3.70 (s, 3H), 4.38(m, 1H), 5.17 (m, 1H), 5.28 (m, 1H), 5.38 (m, 1H), 5.50 (s, 2H), 5.75 (m, 1H), 6.00 (m, 1H), 7.05-7.60 (m, 12H), 7.80 (m, 2H).

### Example 2

### Telmisartan with Lovastatin (FIG. 4)

Following the procedure of EXAMPLE 1, the compound of telmisartan (100 mg) with lovastatin (56 mg) was prepared in a mixture of dichloromethane (1.5 ml) and acetonitrile (0.5 ml) using EDCI (38 mg) as a condensing agent in a presence of catalytical amount of DMAP. Chromatographic purification of a crude reaction mixture afforded 46 mg of the pure compound.

¹H-NMR (CDCl₃), 0.72 (t, 3H), 0.82 (m, 3H), 1.10 (m, 9H), 2.78 (s, 3H), 3.02 (m, 2H) 3.82 (s, 3H), 4.35 (m, 1H), 4.61 (m, 1H), 5.18 (m, 1H), 5.33 (m, 1H), 5.52 (s, 2H), 5.77 (m, 1H), 6.00 (m, 1H), 7.05-7.60 (m, 12H), 7.80 (m, 2H).

### Example 3

### Dimer of Lovastatin with Triethylene Glycol

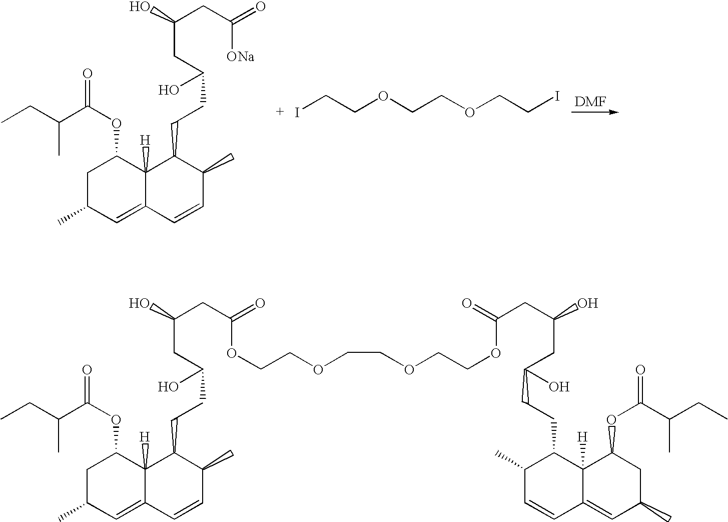

Sodium salt of lovastatin (0.173 g, 0.3892 mmole) and 1,2-bis(iodoethoxy)ethane (0.072 g, 0.1946 mmole) were dissolved in 1.5 mL of anhydrous dimethylformamide at room temperature under argon. The resulting yellow solution was stirred at room temperature in darkness for 48 hr. The reaction mixture was evaporated to dryness and the residue was dissolved in ethyl acetate. The solution was washed with water, brine and dried over anhydrous sodium sulfate. The pure product (0.108 g, 58%) was obtained by chromatographic purification.

### Example 4

### Dimer of Atorvastatin with Diethylene Glycol Bis(glycolate)

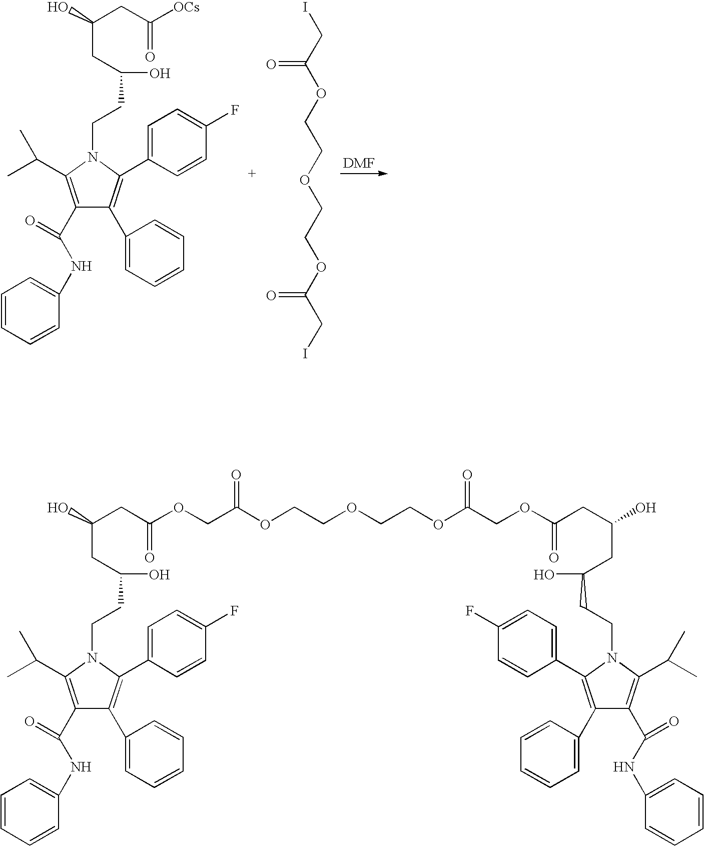

Cesium salt of atorvastatin (0.120 g, 0.2123 mmole) and diethylene glycol bis(iodoacetate) (0.047 g, 0.106 mmole) were dissolved in 1.5 mL of anhydrous dimethylformamide at room temperature under argon. The resulting solution was stirred at room temperature in darkness for 24 hr. The reaction mixture was evaporated to dryness and separated by chromatography on silica gel to afford the product as colorless oil (0.083 g, 60%).

### Example 5

### Dimer of Atorvastatin with Dihydroxyacetone

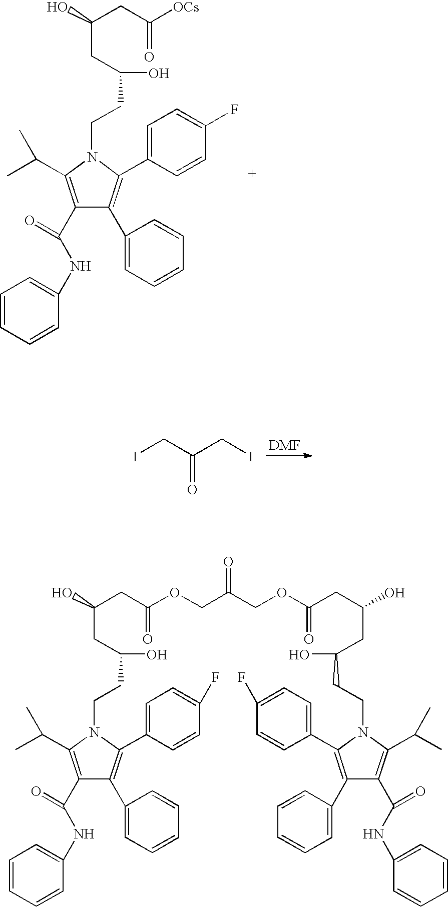

Cesium salt of atorvastatin (0.183 g, 0.3238 mmole) and 1,3-diiodoacetone (0.050 g, 0.1619 mmole) were dissolved in 2 mL of anhydrous dimethylformamide at room temperature under argon. The resulting yellow solution was stirred at room temperature in darkness for 24 hr. The reaction mixture was evaporated to dryness and the residue was dissolved in ethyl acetate. The organic solution was washed with aqueous sodium bisulfite, water and brine. Drying over magnesium sulfate followed by solvent evaporation afforded the crude product, which was purified by preparative TLC chromatography. Yield: 0.098g, 52%.

### Example 6

### Dimer of Atorvastatin with Dihydroxyacetone Bis(glycolate)

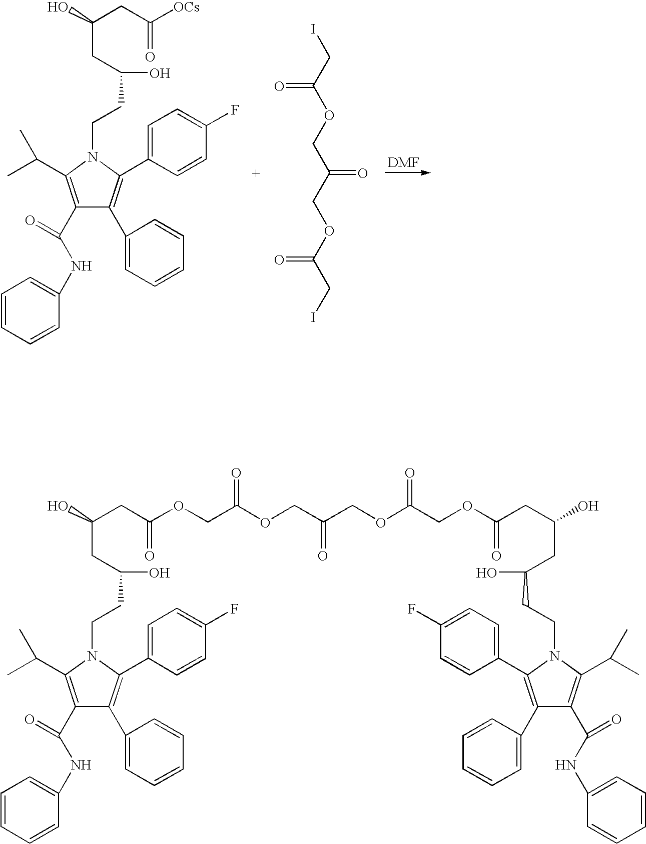

Cesium salt of atorvastatin (0.217 g, 0.3850 mmole) and dihydroxyacetone diiodoacetate (0.082 g, 0.192 mmole) were dissolved in 3 mL of anhydrous dimethylformamide at room temperature under argon. The resulting yellow solution was stirred at room temperature in darkness for 24 hr. The reaction mixture was evaporated to dryness and the residue was dissolved in ethyl acetate. The organic solution was washed with aqueous sodium bisulfite, water and brine. Drying over magnesium sulfate followed by solvent evaporation afforded the crude product, which was purified chromatography. Yield: 0.151 g, 61%.

### Example 7

### Dimer of Atorvastatin with 2,6-Bis(hydroxymethyl)pyridine

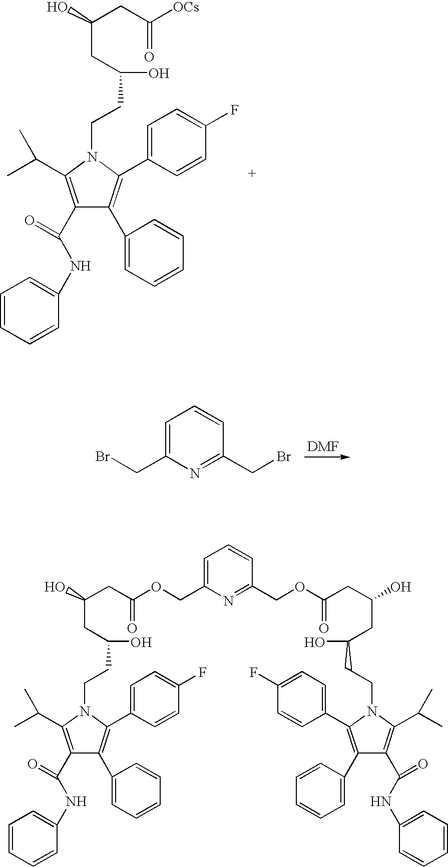

Cesium salt of atorvastatin (0.200 g, 0.3539 mmole) and bis(bromomethyl)pyridine (0.047 g, 0.177 mmole) were dissolved in 2 mL of anhydrous dimethylformamide and 1.5 mL of acetonitrile at room temperature under argon. The resulting colorless solution was stirred at room temperature overnight. The reaction mixture was evaporated to dryness and the residue was dissolved in ethyl acetate. The organic solution was washed with water and brine followed by drying over magnesium sulfate. The crude product was purified by chromatography on silica gel to give 0.119 g, 55% of the dimer.

### Example 8

### Conjugate of Atorvastatin with Ezetimibe

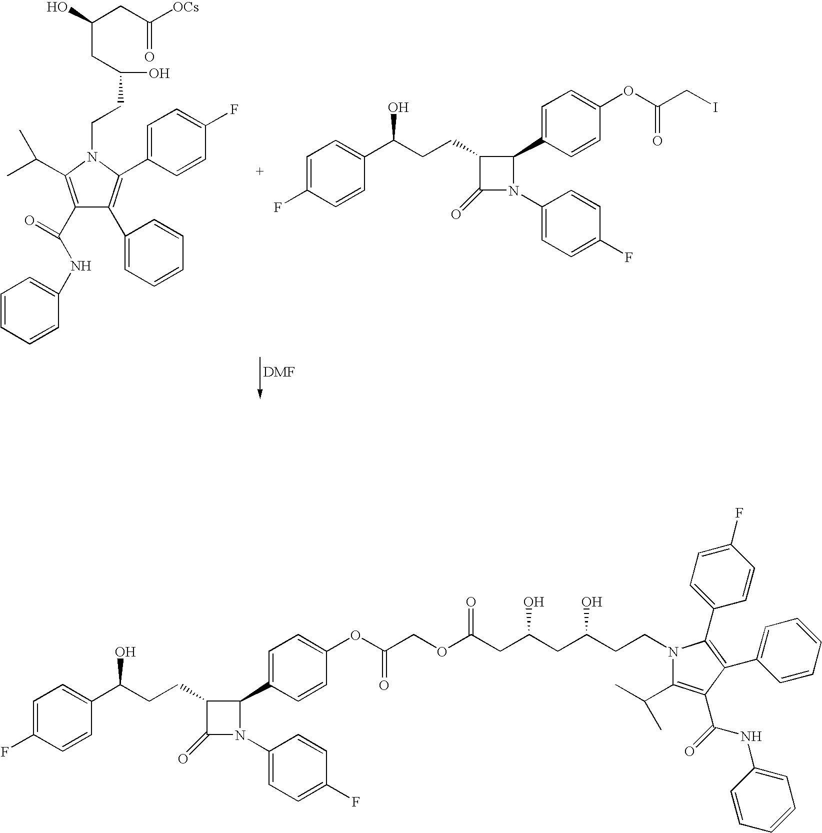

Cesium salt of atorvastatin (0.043 g, 0.0769 mmole) and ezetimibe iodoacetate (0.044 g, 0.0769 mmole) were dissolved in 2.5 mL of anhydrous dimethylformamide at room temperature under argon. The resulting pale yellow solution was stirred at room temperature overnight. The reaction mixture was evaporated to dryness and purified by preparative TLC chromatography to afford 0.042g, (55%) of the conjugate.

### Example 9

### Conjugate of Simvastatin with Ezetimibe

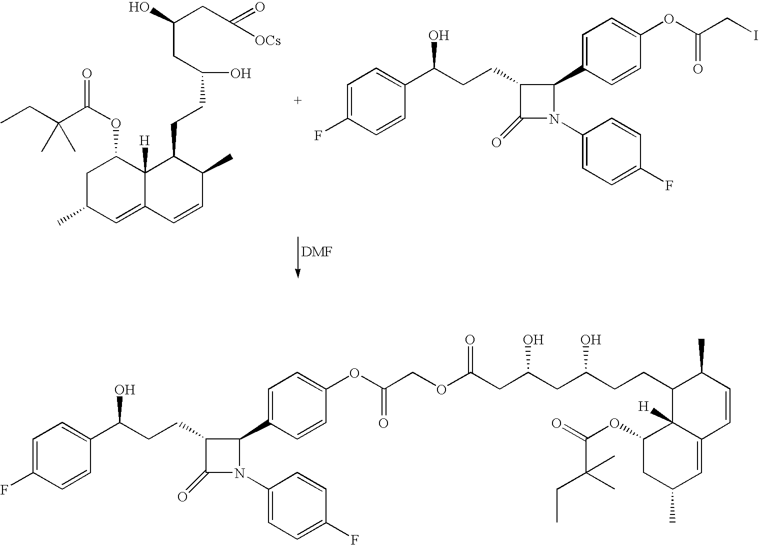

Cesium salt of simvastatin (0.076 g, 0.1337 mmole) and ezetimibe iodoacetate (0.077 g, 0.1337 mmole) were dissolved in 2.5 mL of anhydrous dimethylformamide at room temperature under argon. The resulting pale yellow solution was stirred at room temperature overnight. The reaction mixture was evaporated to dryness and purified by preparative TLC chromatography to afford 0.068g (58%) of the conjugate.

The foregoing description has been presented only for the purposes of illustration and is not intended to limit the invention to the precise form disclosed, but by the claims appended hereto.

All references, patents, and other documents cited herein are expressly incorporated by reference.

We claim:

1. A compound comprising a first pharmacological moiety covalently linked to a second pharmacological moiety through a physiologically labile linkage, or a salt thereof,
(a) wherein the first pharmacological moiety is an HMGCoA reductase inhibitor or a prodrug of an HMGCoA reductase inhibitor; and
(b) wherein the second pharmacological moiety is an angiotensin II (AT ,) receptor blocker or a prodrug of an angiotensin II receptor blocker; a cholesterol absorption inhibitor, a prodrug of cholesterol absorption inhibitor, a cholesteryl ester transfer protein inhibitor, or a prodrug of a cholesteryl ester transfer protein inhibitor; or an HMGCoA reductase inhibitor or a prodrug of an HMGCoA reductase inhibitor.

2. The compound of claim 1, wherein the compound, when exposed to physiologic fluids, decomposes to form an HMGCoA reductase inhibitor and an angiotensin II receptor blocker.

3. The compound of claim 1, wherein the HMGCoA reductase inhibitor is selected from atorvastatin, pravastatin, simvastatin, lovastatin, fluvastatin, cerivastatin, and rosuvastatin.

4. The compound of claim 1, wherein the angiotensin II (AT₁) receptor blocker is selected from telmisartan, losartan, valsartan, irbesartan, candesartan, cilexetil, and other angiotensin II receptor blockers.

5. The compound of claim 1, wherein the compound contains the first pharmacological moiety and the second pharmacological moiety in equimolar amounts.

6. The compound of claim 1, wherein the first pharmacological moiety is covalently linked to the second pharmacological moiety through one or more physiologically labile covalent linkages selected from amide, carbonate, carbamate, ether, ester, sulfonate, and sulfamate bonds.

7. The compound of claim 1, wherein the compound is a mineral acid salt, a carboxylic acid salt, or an amino acid salt.

8. The compound of claim 1, wherein an active drug is regenerated upon cleavage of a covalent bond between the first pharmacological moiety and the second pharmacological moiety.

9. The compound of claim 1, wherein a prodrug is produced upon cleavage of a covalent bond between the first pharmacological moiety and the second pharmacological moiety.

10. The compound of claim 1, wherein an active metabolite is produced upon cleavage of a covalent bond between the first pharmacological moiety and the second pharmacological moiety.

11. An injectable composition comprising the compound of claim 1.

12. The composition of claim 11, wherein the composition liposomes, suspensions, microspheres or nanoparticles.

13. The compound of claim 1, in a solid form.

14. A composition suitable for systemic administration, comprising the compound of claim 1.

15. The composition of claim 14, wherein the composition is selected from capsules, tablets, and gelcaps.

16. A composition suitable for topical administration, comprising the compound of claim 1.

17. The composition of claim 16, wherein the composition is selected from a transdermal patch, ointment, cream, suspension, liquid, elixir and eye drop.

18. An implantable device comprising the compound of claim 1.

19. The device of claim 18, wherein the compound is coated on an implantable device.

20. A composition comprising the compound of claim 1 and an erodible delivery vehicle.

21. A composition comprising the compound of claim 1 and a nonerodible delivery vehicle.

22. A method of treating cardiovascular disease, comprising administering to an individual having cardiovascular disease a pharmaceutically effective amount of compound comprising a first pharmacological moiety covalently linked to a second pharmacological moiety through a physiologically labile linkage, or a salt thereof,
(a) wherein the first pharmacological moiety is an HMGCoA reductase inhibitor or a prodrug of an HMGCoA reductase inhibitor; and
(b) wherein the second pharmacological moiety is an angiotensin II receptor blocker or a prodrug of an angiotensin II receptor blocker; a cholesterol absorption inhibitor, a prodrug of cholesterol absorption inhibitor, a cholesteryl ester transfer protein inhibitor, or a prodrug of a cholesteryl ester transfer protein inhibitor; or an HMGCoA reductase inhibitor or a prodrug of an HMGCoA reductase inhibitor.

23. The method of claim 22, wherein the compound is administered by a method selected from injection, inhalation, implantation, applied as a nasal spray, applied rectally, applied vaginally, ingested orally, and applied topically.

24. The compound of claim 1, wherein the first pharmacological moiety is covalently linked to the second pharmacological moiety through one or more physiologically labile covalent linkages selected from —OCH₂C(O)CH₂O—, —OCH₂CH(OH)CH₂O—, —OCH₂C(O)(OCH₂CH₂)ₙOC(O)CH₂O—, —O(CH₂)₃O—, —(OCH₂CH₂)ₙO—, 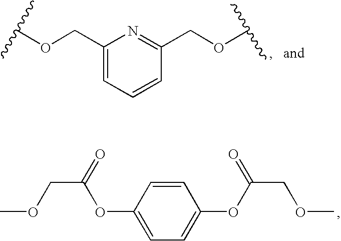
wherein n is an integer from 1 to 6.

25. A compound comprising a first pharmacological moiety covalently linked to a second pharmacological moiety through a physiologically labile linkage, or a salt thereof,
wherein the first and second pharmacological moieties are independently selected from ACE inhibitors, cardioprotective agents, steroids and corticosteroids, sex steroids, apoptosis inhibitors, agents that alter expressions/activity of MMPs, and anti-inflammatory agents.

26. The compound of claim 25, wherein the compound contains the first pharmacological moiety and the second pharmacological moiety in equimolar amounts.

27. The compound of claim 25, wherein the first pharmacological moiety is covalently linked to the second pharmacological moiety through one or more physiologically labile covalent linkages selected from amide, carbonate, carbamate, ether, ester, sulfonate, and sulfamate bonds.

28. The compound of claim 25, wherein the first pharmacological moiety is covalently linked to the second pharmacological moiety through one or more physiologically labile covalent linkages selected from —OCH₂C(O)CH₂O—, —OCH₂CH(OH)CH₂O—, —OCH₂C(O)(OCH₂CH₂)ₙOC(O)CH₂O—, —O(CH₂)₃O—, —OCH₂CH₂)ₙO—, 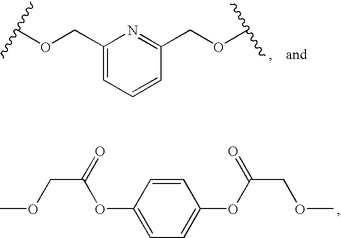
wherein n is an integer from 1 to 6.

29. The compound of claim 25, wherein the compound contains the first pharmacological moiety and the second pharmacological moiety in equimolar amounts.

30. The compound of claim 25, wherein the compound is a mineral acid salt, a carboxylic acid salt, or an amino acid salt.

31. The compound of claim 25, wherein an active drug is regenerated upon cleavage of a covalent bond between the first pharmacological moiety and the second pharmacological moiety.

32. The compound of claim 25, wherein a prodrug is produced upon cleavage of a covalent bond between the first pharmacological moiety and the second pharmacological moiety.

33. The compound of claim 25, wherein an active metabolite is produced upon cleavage of a covalent bond between the first pharmacological moiety and the second pharmacological moiety.

34. An injectable composition comprising the compound of claim 25.

35. The composition of claim 34, wherein the composition liposomes, suspensions, microspheres or nanoparticles.

36. The compound of claim 25 in a solid form.

37. A composition suitable for systemic administration, comprising the compound of claim 25.

38. The composition of claim 37, wherein the composition is selected from capsules, tablets, and gelcaps.

39. A composition suitable for topical administration, comprising the compound of claim 25.

40. The composition of claim 39, wherein the composition is selected from a transdermal patch, ointment, cream, suspension, liquid, elixir and eye drop.

41. An implantable device comprising the compound of claim 25.

42. The device of claim 41, wherein the compound is coated on an implantable device.

43. A composition comprising the compound of claim 25 and an erodible delivery vehicle.

44. A composition comprising the compound of claim 25 and a nonerodible delivery vehicle.

45. A method of treating cardiovascular disease, comprising administering to an individual having cardiovascular disease a pharmaceutically effective amount of compound comprising a first pharmacological moiety covalently linked to a second pharmacological moiety through a physiologically labile linkage, or a salt thereof,
wherein the first and second pharmacological moieties are independently selected from ACE inhibitors, cardioprotective agents, steroids and corticosteroids, sex steroids, apoptosis inhibitors, agents that alter expressions/activity of MMPs, and anti-inflammatory agents.

46. The method of claim 45, wherein the compound is administered by a method selected from injection, inhalation, implantation, applied as a nasal spray, applied rectally, applied vaginally, ingested orally, and applied topically.
